(19) United States
(12) Patent Application Publication
(10) Pub. No.: US 2008/0020443 A1
(43) Pub. Date: Jan. 24, 2008

(54) SIMULTANEOUS CLONING OF GENES AND EXPRESSION OF RECOMBINANT PROTEINS IN ESCHERICHIA COLI

(75) Inventors: Reyes Candau, Olney, MD (US); Robert A. Donahue, Falls Church, VA (US); John Seed, Ellicott City, MD (US)

Correspondence Address: BANNER & WITCOFF, LTD., 1100 13th STREET, N.W., SUITE 1200, WASHINGTON, DC, 20005-4051, US

(73) Assignee: EDGE BIOSYSTEMS, Gaithersburg, MD (US)

(21) Appl. No.: 11/674,596

(22) Filed: Feb. 13, 2007

Related U.S. Application Data
(60) Provisional application No. 60/772,925, filed on Feb. 14, 2006.

(51) Int. Cl. C12N 1/21

(52) U.S. Cl. 435/252.33

(57) ABSTRACT

The mutation of the endA and recA genes in E. coli strain BL21 creates features that are very attractive for protein expression and cloning in a single prokaryotic system, including rapid cell growth, high transformation efficiency, rapid plasmid replication to high densities, and high plasmid stability. When specially prepared for competence, these strains are highly useful for both cloning and protein expression.

This application claims the benefit of and incorporates by reference the contents of provisional application Ser. No. 60/772,925 which was filed on Feb. 14, 2006.

### FIELD OF THE INVENTION

The present invention relates to processes for the preparation of competent cells and bacterial strains for cloning and expression of recombinant proteins in Escherichia coli.

### BACKGROUND OF THE INVENTION

The use of Escherichia coli (E. coli) for the cloning and expression of genes and their encoded proteins has been a central part of molecular biology for many years. In 1970 Mandel and Higa (J. Mol. Biol. 53: 159-162) found that treatment with calcium chloride allowed E. coli to take up bacteriophage lambda DNA. Then in 1972, Cohen et al. (Proc. Natl. Acad. Sci., 69: 2110-2114) showed that these cells could take up plasmid DNA as well. Methods for incorporating plasmid DNA into E. coli were later improved upon and summarized by Hanahan (J. Mol. Biol. 166: 557-580, 1980) and again by Liu & Rashidbaigi (BioTechniques 8: 21-25, 1990). A fairly comprehensive analysis and review of the literature on methods for preparing competent cells in E. coli (cells capable of incorporating and expanding exogenous DNA) and methods of transformation (inserting exogenous DNA into microbial cells) can be found in U.S. Pat. 6,274,369 and is incorporated herein by reference.

The great majority of cells used for bacterial transformation of E. coli are derivatives of strain K-12. This derives in part from the extensive knowledge of the genotype of the K-12 strain, the lack of appropriate genotypes in other strains, and the fact that attempts to render other strains competent with the high levels of transformation efficiency currently required in the practice of cloning have been largely unsuccessful. This is discussed more completely in U.S. Pat. 6,709,852.

While E. coli K-12 derivatives are widely used in cloning, E. coli B strains are typically used to express proteins. The most widely used of these is E. coli strain BL21 and its lysogenic derivative DE3. BL21 is a fast growing Ion and ompT protease deficient bacterial strain. The low intrinsic protease activity of BL21 makes it valuable for expression of recombinant proteins. The principle advantage to BL21(DE3) is that it contains the gene coding for T7 polymerase, an RNA polymerase with high activity. Originally described by Studier & Moffatt (J. Mol. Biol. 189: 113-121, 1984), the gene for T7 RNA polymerase was inserted into the chromosome of BL21 under the control of the lacUV5 promoter (U.S. Pat. Nos. 4,952,496 and 5,869,320). Addition of IPTG activates the lacUV5 promoter, which then drives expression of the T7 polymerase, which then drives the expression of any genes under the control of a T7 promoter. Since there are no endogenous T7 promoters in E. coli, this permits a reasonably good level of control over the expression of recombinant proteins whose DNA sequences are under the control of a T7 promoter after they are inserted into BL21(DE3) cells. Other derivatives of BL21 have been developed that are deficient in recA or are deficient in endA or that contain genes that express rare t-RNAs, a gene that enhances disulfide formation, and others (Novagen Catalog, 2006; Stratagene Catalog 2006).

There is a need in the art to combine all of the cloning features of the K-12 strains with the expression features of BL21 strains. Perhaps this has not been tried due to the intrinsically low transformation efficiency of the B strains. Derivatives of E. coli K-12 strains have been created that offer inducible expression control with T7 polymerase (DE3) and other features common to BL21 protein expression strains. However, these strains lack the convenient Ion and ompT genotypes and the fast growth rates of B21. Thus there is a need in the art for an improved derivative of E. coli strain BL21 that has transfection efficiencies comparable with K-12 strains.

### SUMMARY OF THE INVENTION

One embodiment of the invention provides an isolated cell of a derivative of E. coli strain BL21 that is mutated in the endA and recA genes. The genes may be deleted, insertionally interrupted, point mutated, etc. For example, facilitated recombination and excision of the target gene sequences can be used to create deletions.

Another embodiment of the invention provides a process that increases the transformation efficiency of endA⁻, and/or recA⁻ strains of BL21. The strains are grown in a vegetable-derived protein hydrolysate which increases their electrocompetence.

### DETAILED DESCRIPTION

Isolated bacterial cells according to this invention are derivatives of E. coli strain BL21. They can be mutated, for example deleted, in the endA gene and/or the recA genes. Any mutation may be used, but insertions, deletions, and nonsense mutations are preferred. The sequences of these genes are well known and may be obtained from various sources, the best known of which is GenBank. With knowledge of these gene sequences and of the flanking regions surrounding these sequences in the bacterial genome, these genes may be mutated, preferably deleted, by a variety of methods known to those of skill in the art. These include homologous recombination, random mutagenesis and insertional mutagenesis among others. Successful mutation, e.g., deletion, of the genes is easily determined by sequencing of the target regions. Other techniques for determining the presence or absence of genes that are known to those of skill in the art also may be used.

The strain may be rendered electrocompetent by standard methods. Unlike enda+ and recA+ versions of E. coli strain BL21 these cells are highly electrocompetent and show transformation efficiencies similar to that of K-12 strains, i.e., greater than 10¹⁰ transformants per ug of test plasmid, where the test plasmid is small (2-3 kb), supercoiled and used in pg quantities for the test of transformation efficiency as shown in Table 1.

The transformation efficiency may be further improved by culturing the cells in protein hydrolysates as described in Example 1. This example serves only to illustrate the phenomenon and should not be construed as limiting to the invention, as other sources of vegetable protein hydrolysates also show this effect.

The high transformation efficiency of the ΔendA, ΔrecA BL21 cells that are preferred for protein expression allows scientists to prepare a representative cDNA library and express proteins from that library in the same strain, thereby eliminating a number of gene transfer steps. This reduces the amount of time required to prepare and screen for recombinant proteins by several days, thereby saving a considerable amount of time and money. The increased competency caused by growth with vegetable protein hydrolysates was observed only for electroporation facilitated transformation. Gene mutation and growth in vegetable protein hydrolysates did not affect the chemically induced competence under the conditions heretofore tested. This, however, has limited impact on screening since chemically competent cells are principally used in the process of gene transfer, where the highest possible transformation efficiencies are not required. Furthermore, the need for chemically competent cells is obviated by the availability of an electrocompetent strain with high transformation efficiency, high plasmid stability, fast growth, low protease activity, and suitable for protein expression.

Appropriate amounts of protein hydrolysate to use may vary. Typically at least 0.1%, 0.2%, 0.5%, 1%, 1.5%, 2%, 4%, 5%, or 10%, can be used in a culture medium.

### EXAMPLE 1

### Preparation of a Highly Electrocompetent E. coli Strain BL21

The genes for recA and endA were deleted from E. coli strain BL21 by the method of Link, et al (J. Bacteriol. 179, 6228-6237, 1997) and the deletions were confirmed by sequencing. The modified strain was inoculated into growth media and grown with shaking for 18 hours overnight at 37° C. The culture media contained 1% yeast extract, 10 mM sodium chloride, 5 mM potassium chloride, 2% sodium succinate and 2% of either Difco tryptone (animal protein hydrolysate), Hi-media veg-hydrolysate, or Sigma hydrolysate (vegetable protein hydrolysate). After 18 hours of culture, the cells were diluted to an OD₆₀₀ of 0.3 and cultured in the same media until they reached a final OD₆₀₀ of 1.5. The cells were then pelleted by centrifugation and washed 3 times with 10% glycerol, concentrated to an OD₆₀₀ of 250 and frozen. The transformation efficiency of these cells was tested by thawing an aliquot of the frozen cells on ice, mixing the cells with 10 pg of supercoiled pUC19 DNA and electroporating the DNA into the cells in a Bio-Rad Gene Pulser at 1.4 kv, 200 ohms and 25 uF with a time constant of 4.5-5.0 ms. The cells were then recovered in SOC for 1 hour at 37° C., diluted and plated on LB-agar containing 100 ug/ml ampicillin. After overnight incubation at 37° C., the number of ampicillin resistant clones was determined to obtain the transformation efficiency, which is expressed as the number of transformants per ug of DNA (Table 1).

TABLE 1
Effect of growth in different types of media on the transformation
efficiency of E. coli strain BL21 deleted for recA and endA.

Culture

| Medium | Dilution | Colonies | | | | Average | Efficiency | SEM |
|---|---|---|---|---|---|---|---|---|
| Medium with | 1.00E−02 | 74 | 76 | 89 | 89 | 82 | 1.64E+10 | 8.12E+09 |
| Difco Tryptone | | | | | | | | |
| Medium with | 1.00E−02 | 230 | 193 | 233 | 223 | 219.75 | 4.40E+10 | 1.83E+10 |
| Hi-media veg- | | | | | | | | |
| hydrolysate | | | | | | | | |
| Medium with | 1.00E−02 | 128 | 127 | 186 | 172 | 153.25 | 3.07E+10 | 3.03E+10 |
| Sigma | | | | | | | | |
| hydrolysate | | | | | | | | |

We claim:

1. An isolated bacterial cell which is a derivative of E. coli strain BL21, said cell comprising a mutation in both endA and recA genes which inactivate the encoded proteins of said genes.

2. The isolated bacterial cell of claim 1 which has a transformation efficiency by electroporation of greater than 10¹⁰ transformants/ug of plasmid DNA.

3. The isolated bacterial cell of claim 1 wherein the mutation in both endA and recA is a deletion mutation.

4. A method for increasing the electrocompetence of bacterial cells which are derived from E. coli strain BL21, said cells having a mutation that inactivates the encoded proteins of genes endA and/or recA, said method comprising:
growing said cells in the presence of vegetable-derived protein hydrolysate.

5. The method of claim 4 wherein the bacterial cell has a mutation in both endA and recA.

6. The method of claim 4 wherein the bacterial cell has a deletion mutation in both endA and recA.

7. The method of claim 4 wherein the bacterial cell has a deletion mutation in recA.

8. The method of claim 4 wherein the bacterial cell has a deletion mutation in endA.
